(19) United States
(12) Patent Application Publication
(10) Pub. No.: US 2002/0099275 A1
(43) Pub. Date: Jul. 25, 2002

(54) Method and medical system for monitoring a patient suffering from epilepsy

(75) Inventors: Volker Schmidt, Erlangen (DE); Werner Striebel, Schwarzenbruck (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 10/056,150

(22) Filed: Jan. 24, 2002

(30) Foreign Application Priority Data
Jan. 25, 2001 (DE) 10103327.3

(51) Int. Cl. A61B 5/00

(52) U.S. Cl. 600/300; 128/920

(57) ABSTRACT

In a method and a medical system for monitoring a patient suffering from epilepsy located outside a medical establishment, at least one measured value of the patient relating to the epilepsy is acquired and is supplied to and analyzed with an analysis device and is stored in a central data bank. An alarm generator triggers an alarm signal of a first type if the analyzed measured value is classified as critical for the state of health of the patient or triggers an alarm signal of a second type if the measured value of the patient fails to arrive.

### BACKGROUND OF THE INVENTION

1. Field of the Invention

The invention is directed to a method and to a medical system for monitoring a patient suffering from epilepsy who is located outside a medical establishment.

2. Description of the Prior Art

Effective treatment of patients suffering from epilepsy by a physician requires patient-specific information that, for example, includes the type of an epileptic attack, the frequency of an epileptic attack, or medication taken by the patient. This is particularly true for patients who are difficult to assess in terms of medication and who repeatedly suffer epileptic attacks.

It has been standard practice for extra-clinical monitoring of a patient suffering from epilepsy to have the patient maintain specific measured values at home representing the patient's state of health and the patient's pathogenesis and to communicate these on paper, for example by telefax, to an attending physician, so that the physician can produce a diagnosis based on the measured values and select a suitable therapy for the patient.

A disadvantage with this procedure is that the physician can only monitor the collaboration of the patient as well as the reliability of the patient as to the determination and communication of the measured values by means of a significant outlay for administration. There is therefore the risk that, particularly given negligent patients, deteriorations of the health status or acute health risks will be recognized too late. It is of course possible to have measured values acquired from patients at home by visiting care services, however, these measured values also frequently arrive too late or not at all at the attending physician due to failures in the transmission of the measured values, due to problems in the patient identification, and due to the high outlay for personnel.

### SUMMARY OF THE INVENTION

An object of the present invention is to provide a method and a medical system of the type initially described wherein the monitoring of a patient suffering from epilepsy outside of a medical establishment is improved.

According to the invention, this object is achieved by a method for monitoring at least one patient suffering from epilepsy, where at least one measured value, usually a number of measured values, of a patient relating to the epileptic condition are acquired outside of a medical establishment, and are communicated to a central data bank and are stored at the data bank. An analysis of the measured value or values subsequently ensues, and an alarm signal of a first type is triggered if at least one of the analyzed measured values is classified as critical for the health status of the patient and an alarm signal of a second type is triggered if the expected measured value of the patient fails to arrive. The invention thus makes it possible to centrally store measured values derived from a patient, to analyze these in automated fashion and, given a deterioration of the health status of a patient or given an acute health risk of a patient, to trigger an alarm signal of a first type that, according to one version of the invention, is automatically communicated to a physician treating the patient or to a medical establishment in order to be able to immediately initiate appropriate measures for treating the patient. In this way, it is assured without significant administrative outlay that the patient can receive a treatment appropriate to the patient's health status in a relatively short time. The triggering of an alarm signal of a second type given the failure of measured values of the patient to arrive makes it possible to urge the patient to acquire the measured values, likewise without significant administrative outlay, this likewise resulting in an improved monitoring of the patient. The alarm signal of the second type triggered given the failure of measured values to arrive can be communicated directly to the patient and/or to a physician in order, to inform the physician that measured values have not arrived, as well as to provide an opportunity to influence the patient to acquire the measured values. The method and medical system also offer the advantage of being able to monitor the health status of a number of patients suffering from epilepsy in the same way.

In an embodiment of the invention, at least one criterion for the triggering of the alarm signal of the first type is prescribed, preferably by an attending physician. Criteria for triggering the alarm signal can be defined that apply only to a specific patient or to a number of patients as well. Such criteria can, for example, be tolerance ranges of the measured values or reference values defined by the physician. In a further version of the invention, a learning expert system undertakes the analysis of the measured values and initiates the triggering of the alarm signal of the first type.

In a preferred embodiment of the invention, the measured values are regularly acquired, whereby, as already mentioned, an alarm signal of a second type is always triggered when anticipated measured values of the patient fail to arrive.

In a further embodiment of the invention, an alarm signal of a third type is triggered if no reaction to the alarm signal of the first or second types ensues within a time window, preferably a prescribable time window. The alarm signal of the third type, given no reaction to the alarm signal of the first type, is communicated to a representative of the physician or the medical establishment, and the communication of the alarm signal of the third type given the failure of measured values to arrive may again be communicated to the physician attending the patient.

The measured value or values may be acquired by using a questionnaire, the questionnaire containing questions about the anamnesis, the general condition, the type of attack, the frequency of attacks, the time of attack, complications, an event that triggers an attack, medications, dosage of the medications or side-effects of the medications. The questionnaire thereby need not necessarily include all of these questions. The questionnaire can be individually adapted to the patient.

The measured value or values can be acquired by the patient on the patient's own responsibility, by a care service or by a call center and are communicated to the data bank for storage via a communication network. The data bank preferably can be accessed via a public communication network. In this way, the patient as well as the call center as well as a care service can, for example by e-mail or by a corresponding access authorization to the data bank upon indication of a patient identification number, add the measured values to a patient file maintained in the data bank. In another version of the invention, the measured values stored in the data bank and/or the analyzed measured values can be interrogated via a communication network, preferably via the Internet. Accordingly, every person having a suitable authorization, for example, a physician or a call center authorized by a physician, can interrogate measured values from the data bank, so that the physician can further analyze the measured value or prepare a diagnosis on the basis of the measured values and determine a therapy. In another embodiment of the invention, inputs relating to the diagnosis and/or the therapy of a patient can be undertaken into a patient file of the data bank via the communication network. The patient file in the data bank, accordingly, not only contains the regularly acquired and analyzed measured values but also diagnoses made by the physician and therapy measures that have been initiated.

### DESCRIPTION OF THE DRAWINGS

FIG. 1 schematically illustrates a medical system for monitoring a patient suffering from epilepsy in an extra-clinical environment, in accordance with the invention.

FIG. 2 schematically illustrates a user interface for the configuration of a central alarm generator in accordance with the invention.

FIG. 3 schematically illustrates a user interface for the configuration of the alarms.

### DESCRIPTION OF THE PREFERRED EMBODIMENTS

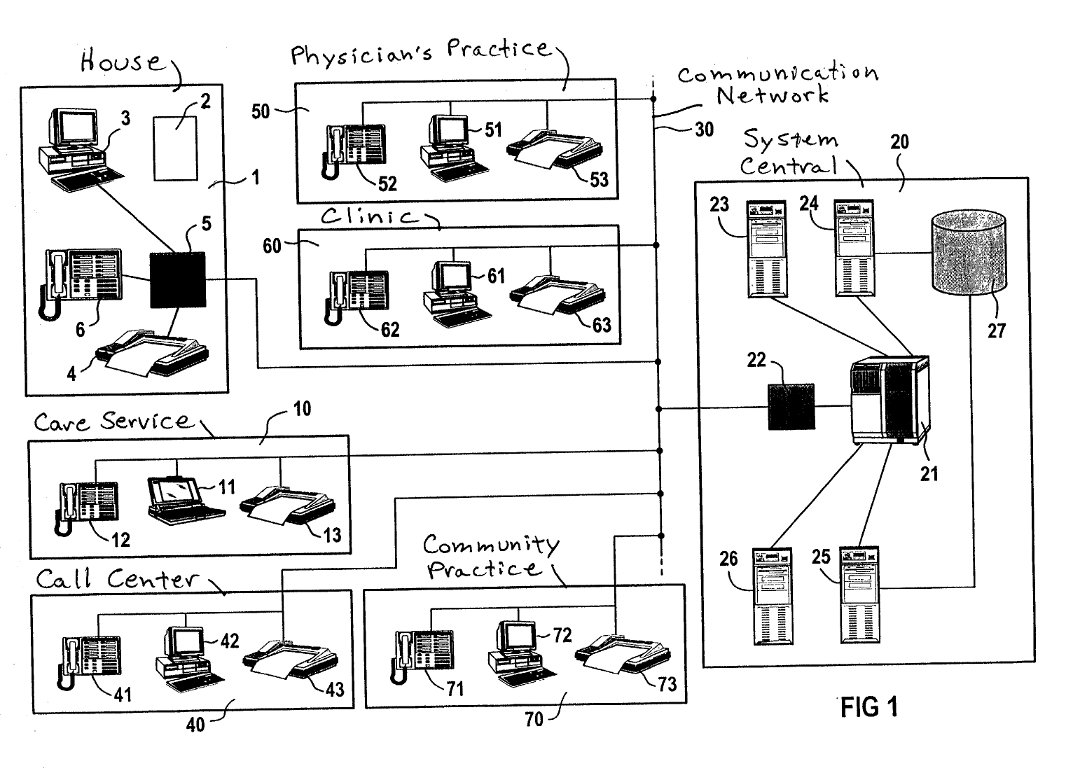

FIG. 1 shows the inventive system for monitoring at least one patient suffering from epilepsy outside a medical establishment. For monitoring the patient, measured values relating to the epilepsy of the patient are acquired. This ensues in the exemplary embodiment by using a questionnaire at the home 1 of the patient, who is not explicitly shown. The questionnaire 2 schematically shown in FIG. 1 contains questions regarding the anamnesis, the general condition of the patient, the type of attack, the frequency of attacks, the time of attack, complications, an event that triggers an attack, medications, dosage of the medications, or side-effects of the medications. The questionnaire can be individually adapted to a patient and can contain other, fewer or even more questions for characterizing the condition of the patient. The questionnaire need not necessarily be in paper form but can be a data file on a computer 3.

The acquisition of the measured values, i.e. filling out the questionnaire whether in paper form 2 or whether at the computer 3, can ensue by the patient on the patient's own responsibility or can be carried out by a caregiver of a care service 10 visiting the patient who can have a computer made available by the care service 10, for example in the form of a lap top 11, for filling out the questionnaire.

The bundle of measured values to be acquired with the questionnaire, preferably at regular points in time, can be communicated to a system center 20 by the computer 3, by a fax machine 4, or by the lap top 11 of the care service 10. This ensues via a communication network, for example via an ISDN network 30, to which, for example, the computer 3 and the fax machine 4 of the patient are connected via an ISDN interface 5. The laptop 11 of the care service 10 likewise can be connected to the communication network 30. The measured data acquired by the care service, however, alternatively can be communicated to the system center 20 by a telephone 12 or by a fax machine 13 via the communication network 30.

Another possibility of acquiring the measured values from the patient is to make use of the services of a call center 40. An employee of the call center 40 acquires the measured data of the patient at regular points in time by telephone 6 or 41 and subsequently communicates the data to the system center 20, likewise by telephone 41, by computer 42 or by fax machine 43 via the communication network 30.

The communication of the measured values by computer preferably ensues in the form of a data file with specification of a patient identification.

The system center 20 serves the purpose of accepting and storing the measured values. Further, it effects a further-processing of the measured values and, when necessary, triggers alarm signals.

To this end, a gateway 21 that is connected to the ISDN network 30 via an ISDN interface 22 is provided in the system center 20. An Internet proxy server 23 for access to the Internet, an analysis device 24 for the measured values, a patient data server 25 for administering the patient data and a communication server 26 as routing device for the collaboration of all components and for routing information can be connected to the gateway 21. A data bank 27 has memory device for the measured values is connected to the analysis device 24 and to the patient data server 25.

The measured values are accepted in the system center 20 via the ISDN interface 22 and are stored long-term in the databank 27. Dependent on the type of communication and form of the measured values, the measured values are converted into a machine-readable data file. The analysis device 24, which can be a commercially available computer, is operated with analysis software that compares the measured values to prescribe criteria stored in the data memory 27 for monitoring patients suffering from epilepsy, and interprets the point in time of the transmission of the measured values. The criteria for the monitoring of a patient suffering from epilepsy or from a number of patients suffering from epilepsy as well as the points in time at which measured values must be present for monitoring the patient or patients can be prescribed. Dependent on where the patient or patients are being treated, the criteria as well as the points in time of the measurements can be prescribed by an independent physician whose practice is referenced 50 in FIG. 1, or can be prescribed by a physician of a medical establishment, for example a clinic 60. The communication of the respective physician with the system center 20 having the data bank 27 wherein the data are kept available in a patient-specific fashion for the analysis device 24, can ensue in a direct way with a computer 51 or 61, with a telephone device 52 or 62 or with a fax machine 53 or 63. The physician or physicians alternatively can make use of the services of the call center 40 for the communication of the prescribed particulars, the call center 40 receiving the prescribed instructions of the physician or physicians by telephone and communicating them to the system center 20.

Criteria for monitoring a patient suffering from epilepsy are represented, for example, by the prescription of tolerance limits for the frequency of occurrence of epileptic attacks or reference values for the taking of medications.

An authorized physician, finally, can directly interrogate the analyzed measured values from the data bank 27, or can have the values interrogated, for example, by the call center 40, and can prepare diagnoses and therapies. To this end, the physician can undertake entries into a patient file stored in the data bank 27, the original and analyzed measured values also being stored therein.

The analysis device 24 also has an alarm generator for generating messages and for triggering alarm signals. All information that are required for triggering alarm signals and, as needed, for forwarding alarm signals and messages given the absence of the intended recipient, are merged in the system center 20. In addition to the physician's practice 50, the clinic 60, the patient, the care service 10 and the call center 40, a practice 70 of a stand-by physician is therefore also connected to the system center 20 via a telephone 71, a personal computer 72, a fax machine 73 or a cell phone (not shown), an alarm signal being able to be forwarded to this stand-by physician in order to initiate measures for treating an affected patient.

An alarm signal of a first type is triggered by the alarm generator given, for example, a significant downward or upward transgression of the measured values of tolerance or reference value limits. The alarm signal of the first type, for example, is communicated to the primary, independent physician or to the primary physician of the clinic 60 via the communication network 30. An attending physician who has authorization can thereby fetch the measured values stored in the data bank 27 and analyzed with the computer 51 or 61, via the telephone 52 or 62, the fax machine 53 or 63, a cell phone or some other communication device in order to initiate suitable measures for the treatment of the patient based on the measured values.

An expert system that is part of the analysis device 24 and that interprets the measured values problem-specifically on the basis of control systems or based on probabilities can be utilized for analyzing the measured values and for triggering alarm signals. An individualization of the expert system to the patient is thereby achieved, i.e. the expert system gets to know the respective patient suffering from epilepsy better and better during the monitoring process; as a learning system, and it constantly makes prognoses about future measured values to be anticipated, comparing these to the true measured values. An individualized monitoring is thus realized.

An alarm signal of a second type is always triggered by the alarm generator when measured values that have become due have not been supplied from the patient. The alarm signal of the second type can be communicated to the patient as a request to send the measured values and also can be communicated to the attending physician in order to indicate the delayed acquisition of the measured values or allow the physician to influence the patient to get the measured values and communicate them to the data bank 27.

An alarm signal of a third type is always triggered when a reaction of an attending physician to an alarm signal of the first type fails to occur within a (preferably prescribable) time window that is dependent on the urgency of the case. Further, an alarm signal of the third type is always triggered when the patient or the physician does not react to an alarm signal of the second type within the time window. The alarm signal of the third type is preferably communicated to the standby physician 70 to allow a decision to be made as to whether to initiate suitable measures or to initiate treatment of the patient.

Dependent on the urgency of a reaction on the part of an informed person or institution, the alarm signals can be generated with different urgency levels, for example utmost urgency, urgent, routine or standard. Which measured values lead to which urgency levels can be defined with the same mechanisms as for the analysis of the measured values and can be stored in the data bank 27. For example, four different tolerance ranges for the measured values can be defined, whereby measured values lying in the first tolerance range trigger an alarm signal of the first type having the level standard, and measured values lying in the fourth tolerance range trigger an alarm signal of the first type having the level of utmost urgency.

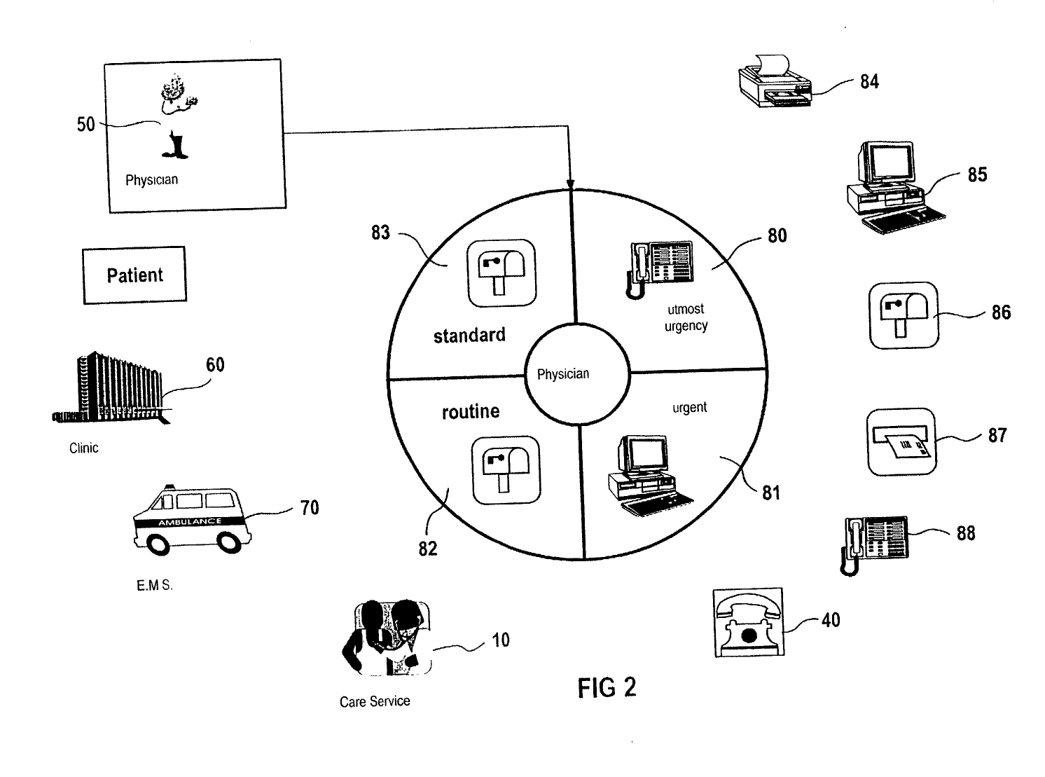

The configuration of the central alarm generator ensues individually by the respective user. Thus, for example, an independently practicing physician defines how he or she wishes to receive messages of different urgency. To this end, this physician uses the user interface shown in FIG. 2. The communication circle is divided into segments 80 through 83 that represent the urgency levels of the alarm messages.

For configuration, the physician utilizes the available information paths or the communication devices connected to the communication network 30 from the information paths that are available, and communication devices 85 through 88 in the corresponding segments 80 through 83 via which the physician would like to be reached given the arrival of a classified message. The configuration is stored in the system center 20.

Given the selected configuration according to FIG. 2, the physician is informed by telephone in cases of utmost urgency, by e-mail via a computer in urgent cases and by regular mail in all other instances.

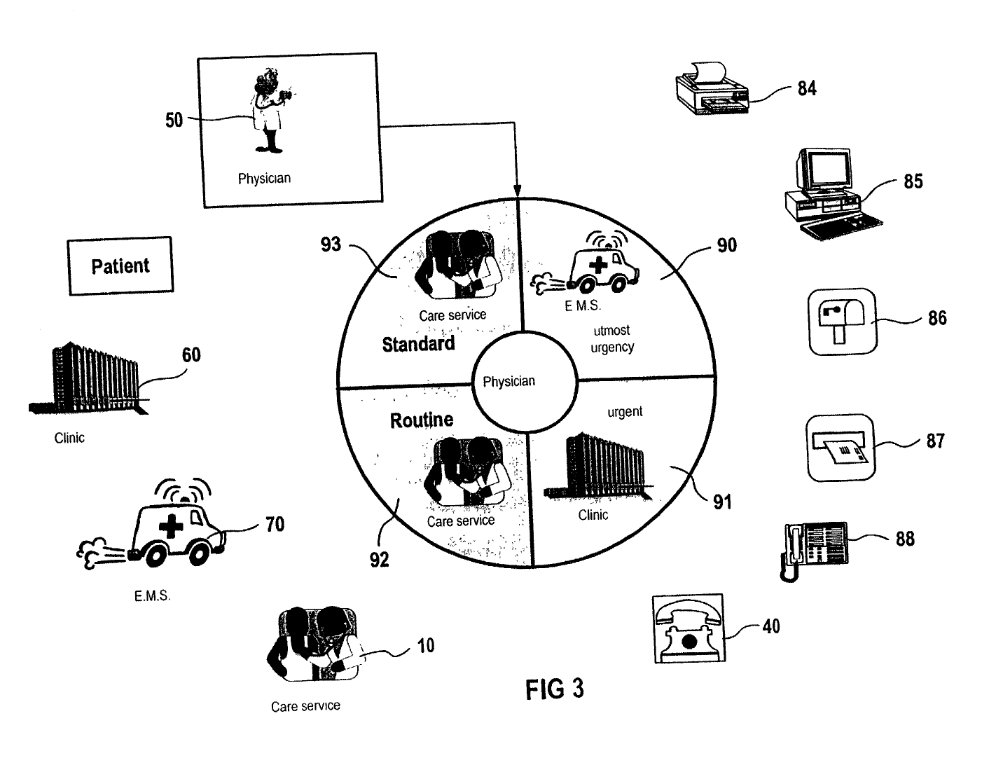

When a physician does not react to an alarm signal of the first or second type within a reasonable time, i.e. within the time window, the alarm generator of the analysis device 24 in the system center 20 triggers an alarm signal of the third type. The configuration regarding the person to whom the alarm signal of the third type should be communicated is undertaken by the attending physician. To this end, the physician employs a communication circle illustrated in FIG. 3 that is segmented on the basis of the urgency levels. The physician drags the institutions 10, 40, 60, 70 into the corresponding circle segments that are to be notified in case of emergency when the physician personally does not react.

Given the selected configuration according to FIG. 3, the standby physician is notified given cases of utmost urgency, the clinic is notified given urgent cases and the care service is notified in all other instances.

The definition of the type of alarm notification to the physician and the prescription of the recipient of the alarm signal of the third type can ensue in a single communication circle.

Default settings can be prescribed by the server, so that the physician is presented with a meaningful proposal given an initial installation, whereby a preference list of the physician is taken into consideration. The information channel to the patient, to the care service, to the clinic or to the standby physician also can be configured as to how the institutions wish to be contacted in the case of the various types of alarm signals and alarm levels. A communication circle into which the available paths are entered is thereby configured that corresponds to the communication circuit shown in FIG. 2.

It is likewise provided that the patient can set a forwarding of urgent messages while on vacation or when in the hospital.

The physicians, patients, care services and standby physicians also can make use of the call center 40 as the transmission path for alarm signals or other information, but the call center 40 must have certain information available, such as regarding the person to whom an alarm signal of the third type is to be forwarded.

The sending of information, for example of alarm signals, occurs such that the analysis device 24 generates an alarm signal and forwards this to the communication server 26 for transmission. As a routing device, the communication server 26 sends the alarm signal to a recipient using a telephone book sub-routine wherein the contact information about an addressable recipient are offered.

The terminal devices employed by the patients, physicians, care services and/or standby physicians can be PDAs (Personal Digital Assistance) with a modem, telephones with voice or dual tone multi-frequency signaling (DTMF), TV set boxes, cell phones, Internet-compatible computer with WWW forms, fax machines or some other device for information transmission. Information such as alarm signals can, accordingly, be communicated via voice output by telephone, via SMS, via e-mail, via WWW, via WWW, via WAP, by fax, by private courier service or by letter mail.

The terminal devices of the patients, physicians, care services and/or standby positions preferably have a graphic user interface based on Internet technologies. For parameterization of the alarm generator, the terminal devices offer the methods visualized in FIG. 2. The method illustrated on the basis of FIG. 3 serves the purpose of parameterizing the notification routes.

In further embodiments, the alarm generator enables triggering of an alarm given measured values that are acquired in a faulty way and triggering of an alarm given compliance problems on the part of the patient.

The analysis device 24 for the measured values and the patient data server 25 can be implemented for data collection so that the measured values are stored reliably and in pseudonym form.

The inventive method and system thus allow monitoring of a patient suffering from epilepsy outside a medical establishment wherein alarm signals are generated given:

Transgression of prescribed limit values (i.e. the measured values lie in an abnormal range)

Faulty patient compliance (anticipated measured values of the patient failed to arrive or the patient does not react to an alarm signal)

Faulty physician compliance (anticipated reaction to an alarm signal by the physician fails to occur).

A configuration design for the reachability and the alarm forwarding enables the settings based on the current needs of each and every treatment case.

Escalation mechanisms see to a forwarding given the lack of a reaction on the part of the physician.

A configuration of the data communication from the physician and/or care giver to the patient can be achieved by the patient.

Although modifications and changes may be suggested by those skilled in the art, it is in the intention of the inventors to embody within the patent warranted hereon all changes and modifications as reasonably and properly come within the scope of their contribution to the art.

We claim as our invention:

1. A method for monitoring an epileptic patient located outside of a medical establishment, comprising the steps of:
acquiring at least one measured value from a patient relating to that patient's epilepsy;
communicating said measured value to a central data bank, remote from said patient, and storing said measured value at said central data bank;
accessing said measured value from said central data bank and analyzing said measured value to obtain an analysis result; and
triggering an alarm signal of a first type if said analysis result is classified as critical for a state of health of said patient, and triggering an alarm signal of a second type if said measured value fails to arrive at said central data bank.

2. A method as claimed in claim 1 comprising transmitting said alarm signal of a first type automatically to a treatment administrator selected from the group consisting of a physician and a medical establishment.

3. A method as claimed in claim 1 comprising selectively prescribing at least one criterion for triggering said alarm signal of said first type.

4. A method as claimed in claim 1 wherein the step of analyzing said measured value comprises analyzing said measured value using a learning expert system, which initiates triggering said alarm signal of said first type.

5. A method as claimed in claim 1 comprising regularly acquiring said measured value.

6. A method as claimed in claim 1 comprising triggering an alarm signal of a third type if no reaction to said alarm signal of said first type or to said alarm signal of said second type occurs within a time window.

7. A method as claimed in claim 1 comprising acquiring said measured value with a questionnaire.

8. A method as claimed in claim 7 comprising asking questions of said patient in said questionnaire to obtain information selected from the group consisting of the patient's anamnesis, the patient's general health condition, a type of epileptic attack suffered by the patient, a frequency of epileptic attacks suffered by the patient, a time of an epileptic attack suffered by the patient, complications experienced by the patient, events which trigger an epileptic attack of the patient, medications being taken by the patient, dosages of medications taken by the patient, and side-effects of medications taken by the patient.

9. A method as claimed in claim 1 wherein the step of acquiring said measured value comprises acquiring said measured value directly from the patient.

10. A method as claimed in claim 1 wherein the step of acquiring said measured value comprises acquiring said measured value via a care service which interacts with the patient.

11. A method as claimed in claim 1 wherein the step of acquiring said measured value comprises acquiring said measured value via a call center in contact with the patient.

12. A method as claimed in claim 1 comprising providing a communication network having access to said data bank to allow retrieval from a remote location of at least one of said measured value stored in said data bank and said analysis result.

13. A method as claimed in claim 1 comprising generating a patient file in said data bank by entering information related to at least one of diagnosis and therapy of said patient via a communication network having remote access to said data bank.

14. A medical system for monitoring an epileptic patient located outside of a medical establishment, comprising:
a data acquisition arrangement for acquiring at least one measured value from a patient relating to that patient's epilepsy;
a communication path;
a central data bank connected to said communication path, remote from said patient, for storing said measured value;
a computer for accessing said measured value from said central data bank and analyzing said measured value to obtain an analysis result; and
said computer triggering an alarm signal of a first type if said analysis result is classified as critical for a state of health of said patient, and triggering an alarm signal of a second type if said measured value fails to arrive at said central data bank.

15. A medical system as claimed in claim 14 wherein said computer transmits said alarm signal of a first type automatically to a treatment administrator selected from the group consisting of a physician and a medical establishment.

16. A medical system as claimed in claim 14 comprising an input unit for selectively prescribing at least one criterion for triggering said alarm signal of said first type.

17. A medical system as claimed in claim 14 wherein said computer contains a learning expert system, which analyzes said measured value and initiates triggering said alarm signal of said first type.

18. A medical system as claimed in claim 14 wherein said data acquisition arrangement regularly acquires said measured value.

19. A medical system as claimed in claim 14 wherein said computer triggers an alarm signal of a third type if no reaction to said alarm signal of said first type or to said alarm signal of said second type occurs within a time window.

20. A medical system as claimed in claim 14 wherein said data acquisition system includes a questionnaire.

21. A medical system as claimed in claim 20 wherein said questionnaire contains questions to obtain information selected from the group consisting of the patient's anamnesis, the patient's general health condition, a type of epileptic attack suffered by the patient, a frequency of epileptic attacks suffered by the patient, a time of an epileptic attack suffered by the patient, complications experienced by the patient, events which trigger an epileptic attack of the patient, medications being taken by the patient, dosages of medications taken by the patient, and side-effects of medications taken by the patient.

22. A medical system as claimed in claim 14 wherein said data acquisition arrangement acquires said measured value directly from the patient.

23. A medical system as claimed in claim 14 wherein said data acquisition arrangement includes a care service which interacts with the patient to obtain said measured value.

24. A medical system as claimed in claim 14 wherein said data acquisition arrangement includes a call center in contact with the patient to obtain said measured value.

25. A medical system as claimed in claim 14 wherein said communication path is a communication network having access to said data bank to allow retrieval from a remote location of at least one of said measured value stored in said data bank and said analysis result.

26. A medical system as claimed in claim 25 comprising an input unit for entering information into said computer related to at lease one of diagnosis and therapy of said patient via said communication network having remote access to said data bank, said computer generating a patient file for said patient including said information.
